(19) United States
(12) Patent Application Publication
(10) Pub. No.: US 2005/0214390 A1
(43) Pub. Date: Sep. 29, 2005

(54) Bioavailability enhancing activity of Carum carvi extracts and fractions thereof

(75) Inventors: Ghulam Nabi Qazi, Jammu and Kashmir (IN); Kasturi Lal Bedi, Jammu and Kashmir (IN); Rakesh Kamal Johri, Jammu and Kashmir (IN); Manoj Kumar Tikoo, Jammu and Kashmir (IN); Ashok Kumar Tikoo, Jammu and Kashmir (IN); Subhas Chandra Sharma, Jammu and Kashmir (IN); Tasaduaq Abdullah, Jammu and Kashmir (IN); Om Parkash Suri, Jammu and Kashmir (IN); Bishan Datt Gupta, Jammu and Kashmir (IN); Krishan Avtar Suri, Jammu and Kashmir (IN); Naresh Kumar Satti, Jammu and Kashmir (IN); Ravi Kant Khajuria, Jammu and Kashmir (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/992,141

(22) Filed: Nov. 18, 2004

Related U.S. Application Data
(62) Division of application No. 10/361,777, filed on Feb. 10, 2003.
Application No. 10/361,777 is a continuation-in-part of application No. 10/200,080, filed on Jul. 19, 2002, now abandoned.
(60) Provisional application No. 60/306,917, filed on Jul. 20, 2001.

(51) Int. Cl. A61K 35/78; A61K 31/497; A61K 31/496; A61K 31/7048

(52) U.S. Cl. 424/725; 424/756; 514/192; 514/28; 514/35; 514/253.08

(57) ABSTRACT

The present invention relates to the use of extracts of Carum carvi as bioenhancers, either alone or in combination with piperine or Zinzeber officinale extract to improve the bioavailability of a wide variety of drugs.

### FIELD OF THE INVENTION

The present invention relates to the use of bioavailability and/or bioefficacy enhancers—also termed as bioenhancers or BE and methods of their preparation which include their isolation from a natural source and obtaining the final products in their chemically characterized fingerprint-profiled form.

The present invention is directed to preparation of active extracts/fractions from the plant Carum carvi which include their chemical characterisation, fingerprint profiling and methods of using such products to enhance bioavailability and/or bioefficacy of drugs, natural products and essential nutraceuticals. The present invention is directed to preparation of composite bioenhancers comprising polar and non-polar extracts of parts of Zingiber officinale and/or piperine (Ex Piper nigrum and Piper longum) which increased significantly (50-180%) the bioavailability of a number of classes of drugs, for example, but not limed to antibiotics, antifungals, anti-virals, anticancer, cardiovascular, CNS, anti-inflammatory/anti-arthritic, anti-TB/anti-leprosy, anti-histaminic/respiratory disorders, corticosteroids, immunosuppressants, anti-ulcer. Such extracts fractions of Carum carvi either in presence OT absence of Zingiber officinale and/or piperine (Ex: Piper nigrum and Piper longum) have been found to be highly selective in their bioavailability bioefficacy enhancing action

### BACKGROUND OF THE INVENTION

There is a great interest and medical need for the improvement of bioavailability of a large number of drugs which are (a) poorly bioavailable, (b) given for long periods, and are (c) toxic and expensive. Maximizing oral bioavailability is therapeutically important base the extent of bioavailability directly influences plasma concentrations and consequently therapeutic efficacy and dose related toxic effects resulting after oral drug administration Poorly bioavailable drugs remain sub-therapeutic because a major portion of a dose never reaches the plasma or exerts its pharmacological effect unless and until very large doses are given which may lead to serious side effects. Any significant improvement in bioavailability will result in lowering the dose or the dose frequency of that particular drug. Besides, inter-subject variability is inversely correlated with the extent of bioavailability. Therefore, low oral bioavailability leads to high variability and poor control of plasma concentration and pharmacodynamic effects. Inter-subject variability is particularly of concern for a drug with a narrow safety margin.

Incomplete oral bioavailability has various causes. These include poor dissolution or low aqueous solubility, poor intestinal membrane permeation, degradation of the drug in gastric or intestinal fluids and pre-systemic intestinal or hepatic metabolism. The normal practice to offset some of these problems has been to increase the dosage as stared earlier which has the concerns of toxicity patients' non-compliance.

Many therapeutic treatments are also accompanied by loss of essential nutraceuticals in the course of therapy. The present invention improves additional status by increasing bioavailability/bioefficacy of various nutraceuticals also which include metals and vitamins. The bioenhancers of the invention also have the potential to enhance the bioefficacy of a drug without influencing its plasma concentrations for various reasons, some of which, but not limited to, are described later in this invention under Section on ‘Bioavailability/Bioenhancing activity’.

### DESCRIPTION OF RELATED ART

Several approaches have been adopted in the past to maximize oral bioavailability, such as (a) particle size reduction (micronization, nanonization, etc.,) (b) polymorphic or crystal size and form selection, (c) solubilization of lesser soluble drugs by way of chemical modifications, complexation and use of co-solvents/surfactants, (d) targeted delivery of drug at the site of action, (e) controlled drug delivery by film coating or use of polymeric matrices for sustained release of drugs, (t) prodrug approach, and (g) microencapsulation using liposomes.

However, based on clues from Ayurvedic literate, a new approach of increasing the bioavailability of drugs including poorly bioavailable drugs had been conceptualized at RRL, Jammu. One of the groups of herbals which has been documented very frequently as essential part of about 70% of Ayurvedic prescriptions, is ‘Trikatu’, that comprises three acrids viz. long pepper, black pepper aid dry ginger in equal proportions. A single major alkaloidal constituent from peppers (piperine) was found to be responsible for bioavailability enhancing effect. The role of ginger is to regulate intestinal function to facilitate absorption Influence of piperine was extensively studied on anti-TB drugs. It was determined that in combination with piperine the dose of rifampicin can be reduced by about 50% while retaining the therapeutic efficacy of His anti-TB drug at par with the standard dose (450 rug). Based on these findings several other reputed plants were evaluated for bioavailability/bioefficacy enhancing activity. Polar and non-polar extracts of parts of a few plants viz., Zingiber officinale, and Cumimun cyminum increased significantly (25-300%), the bioavailability of a number of classes of drugs, for example, but not limited to, antibiotics, antifungals, anti-virals, anticancer, cardiovascular, CNS, anti-inflammatory/anti-arthritic, anti-TB/antileprosy, anti-histaminic/respiratory disorders, corticosteroids, immunosuppressants, anti-ulcer. Such extracts either in presence or absence of piperine have been found to be highly selective in their bioavailability/bioefficacy enhancing action. Carum carvi is a prized culinary herb ad is used extensively in India. The herb finds frequent mention in Ayurvedic and other Indian Systems of Medicine prescriptions against a wide variety of ailments. It remained a scientific curiosity that a single plant can have biological activities for such a large variety of ailments or diseases for which these prescriptions are employed.

Chemistry of Carum carvi

Carum carvi Linn seeds are known as Jira (Beng.), Shahjiru (Guj.), Kala jira, Shiajira (Hindi), Shalajira (Mar.) Carum carvi is an annual or biennial glabrous herb, 30-100 cm height, native to Europe and West Asia, found growing wild in Himachal Pradesh and cultivated in the hills and plains of North India and in the hills of South India or its aromatic seeds.

Its seeds are widely used as a spice for culinary purposes and for flavouring biscuits, cakes, candies, cheese, curries, pickles, sausages, meat products, confectionery and liqueurs of kummel type. They are also used as a flavouring constituent in cordials ad in certain preparations of Cannabis. In medicine, they are used as carminative, mild stomachic, aromatic and diuretic. Both the seeds and the essential oils (caraway oil) are prescribed in flatulent colic and stomach derangements. Exposing the affected parts in patients ring from lumbago and rheumatism to the vapours from the seeds gives relief from the disease. The alcoholic extract of the fruits show dose-dependent antispasmodic effect. Its water finds use as a vehicle for paediatric medicines. Hexane extract of the fruits was found to have excellent larvicidal activity against the mosquito Culex pipiens fatigans Wiedm. [Marketing of Minor Spices in India, 1968, 119; Krishna & Badhwar, J. Sci. Industr. Res., 1952, 11A, suppl., 259; Sharma and Kapil, Loc. cit.; Embong et al Canad J Pl Sci., 1977, 57, 543; I. P., 1966, 104; 1 PC., 55; Gharat, Pharmaceutist, 1958-59, 4 (2), 21; Cappelletti al., J. Ethnopharmacol, 1982, 6, 178; Forster et al, Planta Med, 1980, 40, 309; Deshrmukh et al, Pesticides, 1982, 16 (12), 7]. The dried crushed seeds, on steam distillation, gave a pale yellow to light brown essential oil (known as caraway oil) with a strong aromatic odour. The oil content of the seeds varies according to the degree of maturity of the seeds. Storage affects the oil content of seeds up to 2.8 percent per annum. All Indian samples of the seeds contained 5.8-8.1 percent of caraway oil. Carvone and the limonene are chief constituents of the oils and its odour and flavour are mainly attributed to them. Other constituents present in the oil are α- and β-pinene and ρ-cymene. Besides the above constituents, camphene, Δ³-carene, dihydrocarvone, β-fenchene, myrcene, α- and β-phellandrene, sabinene, α and γ-terpinene, α-thujene, terpinolene, tricyclene, d-and l-dihydropinol, l-neodihydrocarveol, l-isodihydrocarveol, carveol, d-dihydrocarveol, acetaldehyde, methyl alcohol, furfural have also been isolated from European caraway oil (Arctander, 124; Dijkstra and Speckmann, loc. cit.; Atal & Sood, Indian J Pharm, 1967, 29, 42; I. P., 1966, 105; Padha et al Parfum u Kosmetik, 1969, 50, 296; Chem Abstr. 1980, 93, 191892; Salveson & Svendsen, Planta Med. 1976, 30, 93, Guenther, IV, 582).

Caraway oil is primarily used like caraway seeds in flavouring several food products, and in medicine as carminative. It is the main ingredient in the scandinavian “Schnapps” and the German “kummel”. It is employed in gargle preparations, toothpaste flavors, chewing gum, candy and as a masking agent in bad tasting pharmaceutical preparations and obnoxious insecticides. It also exhibits neurotropic anti-spasmodic activity. In mixture with alcohol and castor oil, it is used for the treatment of scabies. The essential oil shows moderate anti-bacterial and anti-fungal property against several bacteria and fungi. Decarvonised oil is sold in the market for scenting cheap soaps, in jasmine bases and tabac perfumes (IPC., 54; Arctander, 125; Chopra et al, 1958, 92; Chem Abstr, 1968, 68, 48218; Narayan et al Indian Drugs, 1979-80, 17, 394; El-keltawi et al, Herba pol, 1980, 26, 245).

The seeds also contain 3-glucosides and 3-galactosides of kaempferol, quercetin and isorhamnetin, and a hydrocarbon (m p, 62-63°). Presence of 5-methoxy-, and 8-methoxy psoralens, sterol, umbelligerone, scopoletin and herniarin is also reported. The fatty acid composition of the oil is: palmitic, 3.6; oleic, 60.7; linoleic, 19.6 and petroselinic, 17.0% (Food technol Abstr., 1974, No. 93, 470; Harborne & Williams, Phytochemistry, 1972, 11, 1741; Ceska et al., ibid, 1987, 26, 165; Chakraborti, Trans Bose Res Inst, 1956-58, 21, 61; Chem. Abstr., 1969, 71, 57561; Hilditch & Williams, 287).

U.S. Pat. No. 5,7441,161 discloses a Zingiber officinale root extract based composition though not as a bioenhancing agent. In addition, piperine has been shown to be active only with certain drugs while showing nil or marginal effects with other drugs in the same therapeutic category. For example, with anti-TB and anti-leprosy drugs, piperine shows enhancement with dapsone and rifampicin to s significant (p, 0.01) to highly significant (<0.001) level respectively. However, it has nil or marginal bioenhancing effect with isoniazid, pyrazinamide and ethambutoL. Similarly, piperine does not enhance the levels of oral hypoglycaemics such as tolbutamide, chlorpropamide.

### OBJECTS OF THE INVENTION

The main object of the invention is to provide a bioavailability enhancing composition containing extracts/fractions of at least Carum carvi.

It is another object of the invention to provide a bioavailability enhancing composition which utilizes the extracts/fractions of Carum carvi with those of either or both Piper nigrum and Zingiber officinale.

It is a further object of the invention to provide a bioefficiency enhancing composition which is a composite of extracts of Carum carvi, Piper nigrum and Zingiber officinale.

### SUMMARY OF THE INVENTION

Accordingly, the present invention provides a bioenhanced composition comprising an effective amount of et and/or bioactive fraction of Carum carvi as a bioavailability enhancer and a therapeutic agent optionally along with an additive or a carrier.

The present invention also provides a composite bioenhancer comprising an effective amount of an aqueous extract or a bioactive fraction of Carum carvi and at least one other bioenhancer useful for enhancing the bioavailability of a drug, neutraceutical, vitamin, antioxidant, natural herbal products and essential nutritional components.

In one embodiment of the invention, the at least one other bioenhancer is selected from piperine and Zingiber officinale extract.

The invention also provides a composition comprising Carum carvi extract, fraction or a mixture thereof piperine and an therapeutically effective amount of a therapeutic agent selected from the group consisting of antibiotic, antimicrobial, antifungal, anti-viral, antitubercular, antileprosy, anti-inflammatory/anti-arthritic, cardiovascular, antihistaminics, CNS drug, respiratory distress relieving drugs, immunosuppressants, antiulcers, nutraceuticals and herbal formulations.

The bioenhancer is preferably used as an aqueous extract or a 50% alcoholic extract from Carum carvi or a fraction thereof or a mixture thereof. The effective dose of the bioenhancer extract used is in the range of 5 to 100 mg/Kg bodyweight. The dose of active fraction of Carum carvi used ranges from 1 to 55 mg.

The antibiotic is selected from the group consisting of fluroquinolone, macrolide, cephalosporin, penicillin and aminoglycoside. The fluroquinolone is selected from the group consisting of ciprofloxacin, o-floxacin and norfloxacin The macrolide is selected from the group consisting of erythromycin, roxythromycin and azithromycin. The cephalosporin is selected from the group consisting of cefadroxil, cefatrioxone, cefixime and cefidinir. The penicillin is selected from amoxycillin and cloxacillin and the aminoglycoside is selected from amikacin and kanamycin. The antifungal agent is selected from the group consisting of fluconazone, amphotericin B and Ketoconazole. The anti-cancer agent is selected from methotrexate and 5-fluorouracil. The cardiovascular agent is selected from the group consisting of lisinopril, atenolol and propranolol. The anti-viral agent is selected from acyclovir and zidovudine. The CNS drug used may be haloperidol. The anti inflammatory/antiarthritic agent is selected from nimesulide and rofecoxib. The anti-TB/antileprosy agent is selected from the group consisting of rifampicin, pyrazinamide, dapsone, etionamide and cycloserine.

The anti-histamines/respiratory disorder agent is selected from the group consisting of salbutamol, theophylline, bromhexine and loratidine. The corticosteroid agent is selected from the group consisting of prednisolone, dexamethasone and betamethasone. The immunosuppressant is selected from cyclosporin A and tacrolimus. The anti-ulcer agent is selected from the group consisting of ranitidine, cimetidine and omeprazole. The herbal extract is selected from the group consisting of extract of Tinospora cordifolia, Picrorrhiza kurroa, Aegles marmelos, Andrographis paniculata, Terminalia chebula, Withania somnifera and Centella asiatica. The neutraceutical agent is selected from the group consisting of vitamin antioxidant, natural herbal product and essential nutritional component.

The vitamin is selected from the group consisting of vitamin A, vitamin E, vitamin B₆ vitamin B₁₂, vitamin C and folic acid. The antioxidant is selected from the group consisting of beta carotene, silymarin and selenium. The essential nutritional component is selected from the group consisting of methionine leucine, lysine, valine, isoleucine, zinc, calcium, glucose, potassium, copper and iron.

The composition is administerable through oral, pare, nasal, inhalation including nebulisers, rectal, vaginal and transdermal routes. The dose of antibiotic ranges from 10-55 mg/kg, that of antifungal agent ranges from 50-80 mg/kg; of anticancer agent used ranges from 5-30 mg/kg; of cardiovascular drug ranges from 0.5-10 mg/kg; the dose of antiviral agent ranges from 10-50 mg/kg; dose of CNS drug ranges from 0.1-0.5 mg/kg; the dose of anti-inflammatory/antiarthritic agent ranges from 2-10 mg/kg; the dose of anti-TB/antileprosy drug ranges from 10-75 mg/kg; the dose of antihiataminics/respiratory drug ranges from 0.5-30 mg/kg; dose of corticosteroid ranges from 0.05-5 mg/kg; dose of immunosuppressant ranges from 5-15 mg/kg. The dose of anti-ulcer agent ranges from 2-45 mg/kg. The dose of vitamin ranges from 0.1 mg/kg-40 mg/kg. The dose of antioxidant ranges from 5 to 15 mg/kg. The dose of essential nutritional component ranges from 20-55 mg/kg. The dose of herbal extract ranges from 10 mg/kg to 1 gm/kg.

Bioavailability/Bioefficacy Enhancing Activity

The present invention relates to the isolation of an exact and/or its Faction from the plant Carum carvi, its standardization with its intended use as drug bioavailability and/or bioefficacy enhancer for the drugs belonging to therapeutic categories such as but not limited to antimicrobial, antifungal, anti-viral, antitubercular, antileprosy, antiinflammatory/anti-arthritic, cardiovascular, antihistaminics, respiratory distress relieving drugs, immunosuppressants, anti-ulcers, nutraceuticals in compositions to be administered orally/parenterally, topically, inhalations (including nebulizers), rely, vaginally in human beings and/or veterinary conditions.

The invention also relates to the preparation of a formulation containing extract and/or its fraction/from the plant Carum carvi and piperine, its standardization with its intended use as drug bioavailability and/or bioefficacy enhancer for the drugs belonging to therapeutic categories such as antimicrobial, antifungal, anti-viral, antitubercular, antileprosy, antiinflammatory, antiarthritic, cardiovascular, antihistaminics, respiratory distress relieving drugs, immunosuppressants, anti-ulcers, nutraceuticals in compositions to be administered orally/parenterally, topically, inhalations (including nebulizers), rectally, vaginally in human beings and/or veterinary conditions.

The invention relates to the preparation of a formulation containing extract and/or its fraction from the plan Carum carvi and Zingiber officinale, its standardization with their intended use as drug bioavailability and/or bioefficacy enhancer for the drugs belonging to therapeutic categories such as antimicrobial, antifungal, anti-viral, antitubercular, antileprosy, antiinflammatory, antiarthritic, cardiovascular, antihistaminics, respiratory distress relieving drugs, immunosuppressants, anti-ulcers, nutraceuticals in compositions to be administered orally/parenterally, topically, inhalations (including nebulizers), rectally, vaginally in human beings and/or veterinary conditions. The bioavailability/bioefficacy enhancer principle may be any extract, its fraction or pure molecule isolated from the plant. Any drug may be selected from the therapeutic categories such as those mentioned above.

The process for the preparation of extract(s)/faction(s) of plants can involve the use of water, alcohol, combinations of water and alcohol, halogenated hydrocarbons ketones ethers as solvents. The plants can include those containing piperine. The composite bioenhancers of the invention having active extracts/fractions from Carum carvi and/or Zingiber officinale with or without piperine make use of physical techniques like dialysis/molecular sieves/membranes, variety of chromatographic techniques and/or liquid-liquid or solid phase extractions, followed by their complete finger prim profiles (HPLC/HPTLC/LC-MS-MS). The combination/s of bioenhancre/s having active extract/fraction do not represent a mere physical mixing but a specialized process for the purpose of formulations that may involve chemical techniques like particle size reduction, use of selective polar solvents or use of ionic/non-ionic surfactants. The formulation of a drug selected from any of the therapeutic categories of the drugs, nutraceuticals, herbal drugs/formulations in combination with the bioenhancer may be intended for routes of administration viz., oral, parenteral, nasal, inhalation including nebulisers, rectal, vaginal, transdermal and others.

The bioenhancing effect of the extracts/fractions of Carum carvi either alone or in combination with extracts/fractions of Zingiber officinale and/or piperine is selective, as shown but not limited to the accompanying examples and does not enhance the bioavailability/bioefficacy of each and every drug, nutraceutical, herbal drug/formulation.

The plant extracts/fractions either individually or in combination express no biological or toxicological effect of their own at the doses at which they are intended to be used.

The aqueous, aqueous—alcoholic, ketonic, ethereal, halogenated solvents eats of the plant parts were evaluated with different therapeutic categories of drugs and nutraceutical (vital amino acids, metals, antioxidants, vitamins) and herbal drugs. The bioavailability/bioefficacy enhancing (BE) activity of Carum carvi exacts was found to be consist from 5 mg to 100 mg irrespective of the amount of the drug (s) present in the formulation. Sub-fractions of the active extracts were also evaluated, with the same categories of drugs. The doses of the fraction (s) responsible for the BE activity ranged from 1.0 to 55 mg. The parent extract as well as the active fraction (s) were found to be active individually as well as in combination with each other with different categories of drugs.

The individual extract or its fractions were found to be 20-110% more active when used in combination with bioenhancer products developed from Zingiber officinale. The effective range for Zingiber officinale. BEs was 10-150 mg. Besides both the parent extracts as well as their fractions from Carum carvi in different combinations showed pronounced activity ranging from 25-95% in presence of piperine. The amount of piperine in these formulations ranged from 3-15 mg.

The extracts or its fractions either in presence or absence of BEs from Zingiber officinale and/or piperine have been found to be highly selective in their bioavailability and/or bioefficacy enhancing activity. This is apparent from the degree of bioavailability and/or bioefficacy enhancement caused by these extracts/fractions as exmeplified in accompanying examples.

The reasons for this selective pattern may be attributable to one or more then one of the following reasons: (a) Promoting the absorption of drugs from GIT, (b) Inhibiting or reducing the rate of biotransformation of drugs in the liver or intestines, (c) Modifying the immune system in a way that the overall requirement of the drug is reduced substantially, (d) Increasing the penetration or the entry into the pathogens even where they become persistors within the macrophages such as for Mycobacterium tuberculosis and such others.

This eventually ensures the enhanced killing of these organisms well secured within the places otherwise inaccessible to the active drug, (e) Inhibiting the capability of pathogens or abnormal tissue to reject the drug erg., efflux mea ms frequently encountered with anti-malarial, anti-cancer and anti-microbial drugs, (f) Modifying the signalling process between host and pathogen erg increased accessibility of the drugs to the pathogens, (g) Enhancing the binding of the drug with the receptors like proteins, DNA, RNA, etc., in the pathogen, thus potentiating and prolonging its effect leading to enhanced antibiotic activity against pathogens, (h) Besides above plausible modes of action, the bioenhancer agents may also be useful for promoting the transport of nutrients and the drugs across the blood brain barrier, which could be of immense help in the control of diseases like cerebral infections, epilepsy and other CNS problems.

Primarily, but not exclusively, the invention enhances the carrier mediated entry of drugs and also the passive diffusion and the active transport pathways in the tissue which are responsible for transporting physiological substances such as nutraceuticals to their target sites. As applicable to any mechanism of action the products of this invention contribute in a synergistic and/or additive manner so that most drugs and nutraceuticals in presence of the products described in the present art are more bioavailable or bioefficaceous as a result of one or more of these mechanisms. The bioavailability and/or bioefficacy of drugs and nutraceuticals is as relevant to animal health besides being important for humans. The invention therefore is also useful in veterinary preparations.

The process for fractionation of the various exams are given in the following schematic representations.

### Flowsheet for Preparation of Extracts of Plant Zingiber officinale

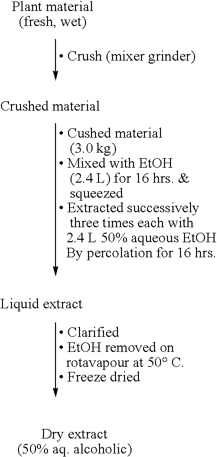

### Flowsheet for Fractionation of Aqueous Extract of Carum carve

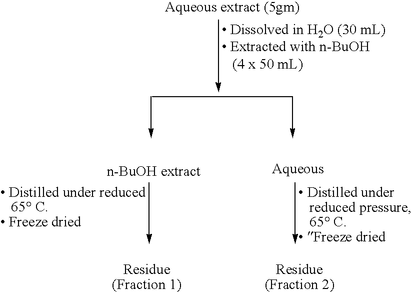

### Flowsheet for Preparation of Extracts of Plant Carum carvi

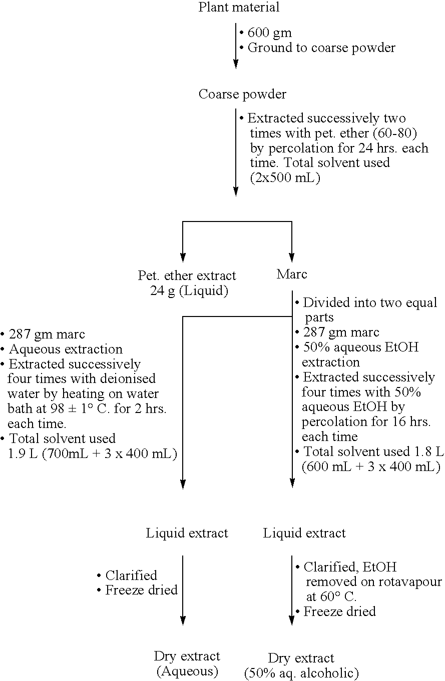

The following examples demonstrate some of the preferred embodiments and should not be construed as limiting the scope of the invention.

TABLE 1
List of drugs as some of the examples for the purpose of the present invention.

| | Categories | Drugs |
|---|---|---|
| I | Antibiotics | Fluoroquinolones: Cipro-, nor-, P-, and O-floxacins |
| | | Macrolides: Erythro-, roxythro-, and Azithromycin |
| | | Cephalosporins: Cefixime, Cefalexin, Cefadroxil and |
| | | Cefatrioxone, cefidinir |
| | | Penicillins: Amoxycillin Cloxacillin |
| | | Aminoglycosides: Amikacin, Kanamycin |
| II. | Antifungal | Fluconazole, Amphotericin B, Ketoconazole |
| III. | Anti-viral | Acyclovir, Zidovudine |
| IV. | Anti-cancer | Methotrexate, 5-Fluorouracil, Dauxorubicin, Cisplatin, |
| | | Adriamycin |
| V. | CVS drugs | Amlodipine, Lisinopril, Atenolol |
| VI. | CNS drugs | Alprazolam Haloperidol |
| VII. | Anti-inflammatory | Diclofenac, Piroxicam, Nimesulide, Rofecoxib |
| | Antiarthritic | |
| VIII | Anti-TB/Antileproxy | Rifampicin, Isoniazid, Pyrazinamide, Ethambutol, Dapsone |
| | drugs | |
| IX. | Anti histamines/ | Salbutamol, Theophylline, Bromhexine, Loretidine |
| | respiratory disorders | |
| X. | Carticosteroids | Prednisolone, dexamethsone, betamethasone |
| XI. | Immunosuppressants | Cyclosporins, Tacrolimus, Mycophenolate mofetil |
| XII | Anti-ulcer | Ranitidine, Cimetidine, Omeprazole |

In all the following tables, bioenhancers of Carum carvi comprise aqueous or 50% alcoholic extract thereof or fraction No. 1. Bioenhancers of Zingiber officinale, mean 50% alcoholic extract of fresh ginger. The doses remain unchanged whether the bioenhancers are used alone or in combination.

### EXAMPLE 1

The amount of bioenhancers used are given below:
i. from Carum carvi: extract 30 mg/kg body weight (Tats); Fraction No. 1: 15 mg/kg body weight (rats)
ii. piperine 8 mg/kg body weight (rats)
iii. Zingiber officinale: 35 mg/kg body weight (rats)

The drug used was rifampicin (40 mg/kg). The drug alone or in combination with the bioenhancers was administered to rats as given below;
Group 1: control
Group 2: Rifampicin alone
Group 3: bioenhancer alone
Group 4: Rifampicin with Carum carvi bioenhancer

Blood from the control/treated animals at predetermined intervals (0-24 hours) (total 14 timings). Rifampicin was extracted from the blood (plasma) using dichloromethane. The concentration of rifapicin in the samples was determined using HPLC (Model: Shimadzu 1080 BP); PDA detector. The mobile phase was phosphate buffer: acetonitrile in a ratio of 40:60, and a flow rate of 1.0 ml/min.

### EXAMPLE 2

The same protocol as in example 1 above was followed for various drugs. The details are given in tables below:

(i) ANTIBIOTICS:

(a) Fluroquinolones

| | | % Enhancement in bioavailability | | | | |
|---|---|---|---|---|---|---|
| | | BE | | | | Carum |
| | Dose | from | Pip- | Carum | BE from | carvi + |
| | mg/ | Carum | erine | carvi + | Zingiber | Zingiber |
| | kg | carvi | as BE | Piperine | officinale | officinale |
| Ciprofloxacin | 45 | 78 | 55 | 110 | 68 | 133 |
| P-floxacin | 40 | Nil | 61 | 70 | 53 | 75 |
| O-floxacin | 20 | 65 | 52 | 167 | 49 | 170 |
| Norfloxacin | 40 | 55 | nil | 65 | Nil | 60 |

(b) Macrolides

| | | % Enhancement in bioavailability | | | | |
|---|---|---|---|---|---|---|
| | | BE | | | | Carum |
| | Dose | from | Pip- | Carum | BE from | carvi + |
| | mg/ | Carum | erine | carvi + | Zingiber | Zingiber |
| | kg | carvi | as BE | Piperine | officinale | officinale |
| Erythromycin | 45 | 70 | 95 | 100 | 68 | 105 |
| Roxythromycin | 15 | 65 | 110 | 95 | 72 | 98 |
| Azithromycin | 25 | 55 | 89 | 90 | 78 | 86 |

(c) Cephalosporins

| | | % Enhancement in bioavailability | | | | |
|---|---|---|---|---|---|---|
| | | BE | | | | Carum |
| | | from | Pip- | Carum | BE from | carvi + |
| | Dose | Carum | erine | carvi + | Zingiber | Zingiber |
| | mg/kg | carvi | as BE | Piperine | officinale | officinale |
| Cefalexin | 45 | Nil | 90 | 90 | 75 | 79 |
| Cefadroxil | 45 | 67 | 70 | 95 | 68 | 85 |
| Cefatrioxone | 25 | 72 | Nil | 78 | Nil | 75 |
| Cefixime | 40 | 80 | nil | 79 | Nil | 82 |
| Cefidinir | 40 | 89 | 60 | 95 | 35 | 130 |

(d) Penicillins

| | | % Enhancement in bioavailability | | | | |
|---|---|---|---|---|---|---|
| | | BE | | | | Carum |
| | | from | Pip- | Carum | BE from | carvi + |
| | Dose | Carum | erine | carvi + | Zingiber | Zingiber |
| | mg/kg | carvi | as BE | Piperine | officinale | officinale |
| Amoxycillin | 45 | 75 | 120 | 115 | 80 | 100 |
| Cloxacillin | 25 | 110 | 87 | 95 | 76 | 110 |

(e) Aminoglycosides

| | | % Enhancement in bioavailability | | | | |
|---|---|---|---|---|---|---|
| | | BE | | | | Carum |
| | | from | Pip- | Carum | BE from | carvi + |
| | Dose | Carum | erine | carvi + | Zingiber | Zingiber |
| | mg/kg | carvi | as BE | Piperine | officinale | officinale |
| Amikacin | 50 | 85 | nil | 100 | Nil | 92 |
| Kanamycin | 50 | Nil | 72 | 87 | 65 | 68 |

(ii) ANTIFUNGAL

| | | % Enhancement in bioavailability | | | | |
|---|---|---|---|---|---|---|
| | | BE | | | | Carum |
| | Dose | from | Pip- | Carum | BE from | carvi + |
| | mg/ | Carum | erine | carvi + | Zingiber | Zingiber |
| | kg | carvi | as BE | Piperine | officinale | officinale |
| Fluconazole | 65 | 65 | 87 | 98 | 120 | 110 |
| Amphotericin | 78 | 78 | nil | 90 | Nil | 80 |
| B | | | | | | |
| Ketoconazole | 55 | 55 | 105 | 100 | 125 | 96 |

(iii) ANTI-CANCER

| | | % Enhancement in bioavailability | | | | |
|---|---|---|---|---|---|---|
| | | BE | | | | Carum |
| | Dose | from | Pip- | Carum | BE from | carvi + |
| | mg/ | Carum | erine | carvi + | Zingiber | Zingiber |
| | kg | carvi | as BE | Piperine | officinale | officinale |
| Methotrexate | 5 | 76 | 65 | 89 | 87 | 102 |
| 5-Fluorouracil | 25 | 90 | 87 | 110 | 110 | 100 |
| Dauxorubicin | 5 | Nil | 68 | 70 | 72 | 69 |
| Cisplatin | 5 | Nil | nil | nil | 56 | 55 |

(iv) CARDIOVASCULAR

| | | % Enhancement in bioavailability | | | | |
|---|---|---|---|---|---|---|
| | | BE | | | | Carum |
| | | from | Pip- | Carum | BE from | carvi + |
| | Dose | Carum | erine | carvi + | Zingiber | Zingiber |
| | mg/kg | carvi | as BE | Piperine | officinale | officinale |
| Amlodipine | 1.0 | Nil | 43 | 50 | 68 | 65 |
| Lisinopril | 1.0 | 79 | 85 | 95 | 76 | 90 |
| Atenolol | 5 | 100 | nil | 93 | Nil | 97 |
| Propranolol | 8 | 68 | 84 | 90 | 76 | 75 |

(v) ANTI-VIRAL

| | | % Enhancement in bioavailability | | | | |
|---|---|---|---|---|---|---|
| | | BE | | | | Carum |
| | | from | Pip- | Carum | BE from | carvi + |
| | Dose | Carum | erine | carvi + | Zingiber | Zingiber |
| | mg/kg | carvi | as BE | Piperine | officinale | officinale |
| Acyclovir | 40 | 78 | 96 | 100 | 82 | 90 |
| Zidovudine | 10 | 92 | 140 | 95 | 105 | 87 |

(VI) CNS DRUGS:

| | | % Enhancement in bioavailability | | | | |
|---|---|---|---|---|---|---|
| | | BE | | | | Carum |
| | | from | Pip- | Carum | BE from | carvi + |
| | Dose | Carum | erine | carvi + | Zingiber | Zingiber |
| | mg/kg | carvi | as BE | Piperine | officinale | officinale |
| Alprazolam | 0.1 | Nil | 65 | 70 | 76 | 80 |
| Haloperidol | 0.5 | 95 | nil | 90 | Nil | 85 |

(vii) ANTI-INFLAMMATORY/ANTIARTHRITIC:

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Diclofenac | 5 | Nil | 120 | 100 | 90 | 95 |
| Piroxicam | 2 | Nil | 110 | 98 | 86 | 76 |
| Nimesulide | 10 | 100 | 132 | 140 | 144 | 145 |
| Rofecoxib | 2.5 | 75 | nil | 70 | Nil | 80 |

(viii) ANTI-TB/ANTILEPROSY DRUGS:

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Rifampicin | 40 | 110 | 45 | 170 | 65 | 140 |
| Isoniazid | 25 | Nil | nil | nil | Nil | nil |
| Pyrazinamide | 12.5 | 45 | nil | 50 | Nil | 55 |
| Ethambutol | 70 | Nil | nil | nil | Nil | nil |
| Dapsone | 10 | 56 | 34 | 67 | 46 | 68 |
| Ethionamide | 25 | 68 | 45 | 65 | 56 | 70 |
| Cycloserine | 40 | 70 | 67 | 80 | 71 | 75 |

(ix). ANTI-HISTAMINES/RESPIRATORY DISORDERS:

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Salbutamol | 0.8 | 75 | 60 | 89 | 78 | 80 |
| Theophylline | 30 | 70 | 65 | 79 | 76 | 89 |
| Bromhexine | 25 | Nil | 67 | 70 | 67 | 71 |
| Loratidine | 1.0 | 76 | nil | 70 | Nil | 80 |

(x) CORTICOSTEROIDS:

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Prednisolone | 4 | 65 | nil | 67 | Nil | 60 |
| Dexamethasone | 0.05 | 72 | 66 | 77 | 76 | 73 |
| Betamethasone | 0.1 | 80 | 72 | 89 | 75 | 77 |

(Xi) IMMUNOSUPPRESSANTS:

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Cyclosporin A | 10 | 100 | nil | 105 | 116 | 120 |
| Tacrolimus | 5 | 90 | 105 | 95 | 75 | 114 |
| Mycophenolate | 15 | Nil | nil | nil | Nil | nil |
| Mofeit | | | | | | |

(Xii) ANTI-ULCER

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Ranitidine | 30 | 67 | 21 | 70 | 147 | 150 |
| Cimetidine | 40 | 72 | nil | 84 | 98 | 100 |
| Omeprazole | 2 | 76 | nil | 70 | nil | 75 |

D. Herbal formulation

| | | % Enhancement in bioavailability | | | | |
|---|---|---|---|---|---|---|
| | | | | | BE from | Carum |
| | Dose | BE from | Piperine | Carum | Zingiber | carvi + Zingiber |
| | mg/kg | Carum carvi | as BE | carvi + Piperine | officinale | officinale |
| Echinacea | 10 | Nil | 75 | 76 | 66 | 65 |
| Augustifolia | | | | | | |
| Tinospora | 50 | 76 | 85 | 90 | 67 | 71 |
| cordifolia | | | | | | |
| Picrorrhiza | 50 | 80 | 78 | 110 | 56 | 76 |
| kurroa | | | | | | |
| Aegles | 1000 | 65 | Nil | 65 | Nil | 60 |
| marmelos | | | | | | |
| Andrographis | 50 | 68 | 63 | 72 | 55 | 54 |
| paniculata | | | | | | |
| Emblica ribes | 50 | Nil | Nil | nil | 65 | 68 |
| Asparagus | 50 | Nil | 58 | 55 | 44 | 45 |
| racemosus | | | | | | |
| Terminalia | 50 | 92 | Nil | 87 | Nil | 91 |
| chebula | | | | | | |
| Withania | 60 | 76 | 55 | 70 | 64 | 76 |
| somnifera | | | | | | |
| Centella | 30 | 68 | nil | 65 | nil | 62 |
| asiatica | | | | | | |

E. Nutraceuticals

| | | % Enhancement in bioavailability | | | | | | | |
|---|---|---|---|---|---|---|---|---|---|
| | | | | | Carum carvi + | | | Carum carvi + | |
| | | Carum carvi | | | piperine | | BE from | Zingiber officinale | |
| | Dose | (Cc) | | Piperine | | Frac 1. | Zingiber | | Frac 1. |
| | mg/kg | Extr. | Frac 1. | as BE | Extr. (Cc) | (Cc) | officinale | Extr. (Cc) | (Cc) |
| Vitamins | | | | | | | | | |
| Vitamin A | 1 mg | 17 | 19 | 11 | 13 | 16 | Nil | 20 | 27 |
| Vit E | 40 mg | Nil | Nil | Nil | Nil | Nil | Nil | Nil | Nil |
| Vit B 1 | 10 mg | 37 | 42 | 17 | 21 | 26 | 31 | 43 | 55 |
| Vit B 6 | 0.5 | nil | Nil | Nil | Nil | Nil | Nil | Nil | Nil |
| Vit B 12 | 0.1 ug | Nil | nil | Nil | Nil | Nil | Nil | Nil | Nil |
| Vit C | 50 mg | Nil | Nil | Nil | Nil | Nil | Nil | Nil | Nil |
| Folic Acid | 50 ug | Nil | nil | nil | nil | nil | nil | nil | nil |
| Antioxidants | | | | | | | | | |
| B-carotene | 15 mg | 47 | 55 | 29 | 45 | 59 | 35 | 63 | 72 |
| Silymarin | 5 mg | 31 | 38 | Nil | 36 | 45 | 33 | 38 | 41 |
| Selenium | 2 ug | Nil | nil | Nil | Nil | nil | Nil | nil | nil |
| Natural Herbal | | | | | | | | | |
| Products | | | | | | | | | |
| Curcumin | 50 mg | 40 | 48 | 39 | 42 | 51 | 49 | 43 | 52 |
| Boswellic acids | 50 mg | Nil | nil | Nil | Nil | nil | Nil | Nil | nil |
| extract | | | | | | | | | |
| Rutin | 40 mg | 34 | 45 | 33 | 36 | 40 | 42 | 34 | 42 |
| Essential | | | | | | | | | |
| nutritional | | | | | | | | | |
| components | | | | | | | | | |
| Methionine | 20 mg | 20 | 28 | 11 | 25 | 30 | 23 | 30 | 37 |
| Lysine | 40 mg | 26 | 29 | 31 | 33 | 38 | 19 | 39 | 43 |
| Leucine | 50 mg | 19 | 21 | 22 | 29 | 32 | 24 | 35 | 40 |
| Valine | 25 mg | 16 | 19 | nil | 22 | 29 | 21 | 32 | 38 |
| Isoleucine | 25 mg | 28 | 34 | 16 | 37 | 44 | 15 | 42 | 50 |
| Zinc* | 0.1 mg | nil | Nil | nil | nil | Nil | nil | Nil | Nil |
| Calcium* | 30 mg | nil | Nil | nil | nil | Nil | nil | Nil | Nil |
| Glucose | 50 mg | 28 | 31 | 35 | 41 | 50 | 13 | 37 | 46 |
| Potassium* | 25 mg | nil | Nil | nil | nil | Nil | nil | Nil | Nil |
| Copper* | 30 mg | nil | Nil | nil | nil | Nil | nil | Nil | Nil |
| Iron* | 0.5 mg | nil | nil | nil | nil | nil | nil | nil | nil |

*Doses equivalent to elemental concentration

1. (canceled)

2. (canceled)

3. (canceled)

4. A bioenhancing composition comprising an effective amount of an extract of Carum carvi, a bioactive fraction of Carcum carvi or a combination thereof as a bioavailability enhancer; piperine and a therapeutically effective amount of a therapeutic agent selected from the group consisting of antibiotics, antimicrobials, antifungals, anti-virals, anti-cancer agents antituberculars, antileprosy agents, anti-inflammatories, anti-arthritics cardiovascular agents, antihistaminics, CNS drugs, respiratory disorder relieving drugs, corticosteroids immunosuppressants, antiulcers agents, nutraceuticals and herbal formulations.

5. (canceled)

6. (canceled)

7. (canceled)

8. The composition as claimed in claim 4, wherein the antibiotic is selected from the group consisting of fluoroquinolone macrolide, cephalosporin, penicillin and aminoglycoside.

9. The composition as claimed in claim 8, wherein the fluoroquinolone is selected from the group consisting of ciprofloxacin, o-floxacin and norfloxacin.

10. The composition as claimed in claim 8, wherein the macrolide is selected from the group consisting of erythromycin, roxythromycin and azithromycin.

11. The composition as claimed in claim 8, wherein the cephalosporin is selected from the group consisting of cefadroxil, cefatrioxone, cefixime and cedifinir.

12. The composition as claimed in claim 8, wherein the wherein the penicillin is selected from the group consisting of amoxycillin and cloxacillin.

13. The composition as claimed in claim 8, wherein the aminoglycoside is selected from the group consisting of amikacin and kanamycin.

14. The composition as claimed in claim 4, wherein the antifungal agent is selected from the group consisting of fluconazone, amphotericin B and Ketoconazole.

15. The composition as claimed in claim 4, wherein the anti-cancer agent is selected from the group consisting of methotrexate and 5-fluorouracil.

16. The composition as claimed in claim 4, wherein the cardiovascular agent is selected from the group consisting of lisinopril, atenolol and propranolol.

17. The composition as claimed in claim 4, wherein the anti-viral agent is selected from the group consisting of acyclovir and zidovudine.

18. The composition as claimed in claim 4, wherein the CNS drug is haloperidol.

19. The composition as claimed in claim 4, wherein the anti inflammatory/antiarthritic agent is selected from the group consisting of nimesulide and rofecoxib.

20. The composition as claimed in claim 4, wherein the antitubercular or/antileprosy agent is selected from the group consisting of rifampicin, pyrazinamide, dapsone, etionamide and cycloserine.

21. The composition as claimed in claim 4, wherein the anti-histamines/or respiratory disorder agent is selected from the group consisting of salbutamol, the ophylline, bromhexine and loratidine.

22. The composition as claimed in claim 4, wherein the corticosteroid agent is selected from the group consisting of prednisolone, dexamethasone and betamethasone.

23. The composition as claimed in claim 4, wherein the immunosuppressant is selected from the group consisting of cyclosporin A and tacrolimus.

24. The composition as claimed in claim 4, wherein the anti-ulcer agent is selected from the group consisting of ranitidine, cimetidine and omeprazole.

25. The composition as claimed in claim 4, wherein herbal formulation is a herbal extract and is selected from the group consisting of an extract of Tinospora cordifolia, Picrorrhiza kurroa, Aegles marmelos, Andrographis paniculata, Terminalia chebula, Withania somnifera and Centella asiatica.

26. The composition as claimed in claim 4, wherein the nutraceutical agent is selected from the group consisting of vitamins, antioxidants, natural herbal products and essential nutritional components.

27. The composition as claimed in claim 26, wherein the vitamin is selected from the group consisting of vitamin A, vitamin E vitamin B₆, Vitamin B₁₂, vitamin C and folic acid.

28. The composition as claimed in claim 26, wherein the antioxidant is selected from the group consisting of beta carotene, silymarin and selenium.

29. The composition as claimed in claim 26, wherein the essential nutritional component is selected from the group consisting of methionine leucine, lysine, valine, isoleucine, zinc, calcium, glucose, potassium, copper and iron.

30. (canceled)

31. The composition as claimed in claim 4, wherein the dose of antibiotic ranges from 10-55 mg/kg.

32. The composition as claimed in claim 4, wherein the dose of antifungal agent ranges from 50-80 mg/kg.

33. The composition as claimed in claim 4, wherein the dose of anticancer agent used ranges from 5-30 mg/kg.

34. The composition as claimed in claim 4, wherein the dose of cardiovascular drug ranges from 0.5-10 mg/kg.

35. The composition as claimed in claim 4, wherein the dose of antiviral agent ranges from 10-50 mg/kg.

36. The composition as claimed in claim 4, wherein the dose of CNS drug ranges from 0.1-0.5 mg/kg.

37. The composition as claimed in claim 4, wherein the dose of anti-inflammatory/antiarthritic agent ranges from 2-10 mg/kg.

38. The composition as claimed in claim 4, wherein the dose of anti-TB/antileprosy drug ranges from 10-75 mg/kg.

39. The composition as claimed in claim 4, wherein the dose of antihiataminics/respiratory drug ranges from 0.5-30 mg/kg.

40. The composition as claimed in claim 4, wherein the dose of corticosteroid ranges from 0.05-5 mg/kg.

41. The composition as claimed in claim 4, wherein the dose of the immunosupressant ranges from 5-15 mg/kg.

42. The composition as claimed in claim 4, wherein the dose of anti-ulcer agent ranges from 2-45 mg/kg.

43. The composition as claimed in claim 4, wherein the dose of vitamin ranges from 0.1 mg/kg-40 mg/kg.

44. The composition as claimed in claim 26, wherein the dose of antioxidant ranges from 5 to 15 mg/kg.

45. The composition as claimed in claim 26, wherein the dose of essential nutritional component ranges from 20-55 mg/kg.

46. The composition as claimed in claim 25, wherein the dose of herbal extract ranges from 10 mg/kg to 1 gm/kg.

47. The composition as claimed in claim 4, wherein the bioavailability enhancement of antibiotic is 50-110%.

48. The composition as claimed in claim 4, wherein the bioavailability enhancement of the antifungal agent ranges between 50-80%.

49. The composition as claimed in claim 4, wherein the bioavailability enhancement of anticancer agent ranges between 70-90%.

50. The composition as claimed in claim 4, wherein the bioavailability enhancement of the cardiovascular drug ranges between 60-100%.

51. The composition as claimed in claim 4, wherein the bioavailability enhancement of the antiviral agent ranges between 70-95%.

52. The composition as claimed in claim 4, wherein the bioavailability enhancement of the CNS drug ranges between 90-95%.

53. The composition as claimed in claim 4, wherein the bioavailability enhancement of the antiinflammatory agent ranges between 70-100%.

54. The composition as claimed in claim 4, wherein the bioavailability enhancement of anti-TB/antileprosy agent ranges between 40 to 110%.

55. The composition as claimed in claim 4, wherein the bioavailability enhancement of the antihistamine ranges between 70-80%.

56. The composition as claimed in claim 4, wherein the bioavailability enhancement of corticosteroid ranges between 60-80%.

57. The composition as claimed in claim 4, wherein the bioavailability enhancement of the immunosuppressant ranges between 80-100%.

58. The composition as claimed in claim 4, wherein the bioavailability enhancement of the anti-ulcer agent ranges between 60-80%.

59. (canceled)

60. (canceled)

61. (canceled)

62. (canceled)
